(19) United States
(12) Patent Application Publication
(10) Pub. No.: US 2016/0311409 A1
(43) Pub. Date: Oct. 27, 2016

(54) BIOLOGICAL SECURITY VEHICLE WASHING APPARATUS AND METHOD

(71) Applicant: Biofence Inc., Alexandria, MN (US)

(72) Inventor: Jeffrey Allen Schutz, Alexandria, MN (US)

(21) Appl. No.: 15/134,899

(22) Filed: Apr. 21, 2016

Related U.S. Application Data
(60) Provisional application No. 62/150,696, filed on Apr. 21, 2015.
(60) Provisional application No. 62/157,657, filed on May 6, 2015.

(51) Int. Cl. B60S 3/04; A61L 2/24; B08B 13/00; A61L 2/22; B05B 13/02; B08B 3/02

(52) U.S. Cl. CPC B60S 3/042; B05B 13/0285; B08B 3/02; B08B 13/00; A61L 2/22; A61L 2/24; A61L 2202/14; A61L 2202/15; A61L 2202/17

(57) ABSTRACT

An automatic vehicle disinfection system for promoting biosecurity through application of a disinfectant to a vehicle transiting to and from an agricultural site. The automatic vehicle disinfection system including a station having one or more of opposed vehicle wash ramps, a disinfectant supply system including a pressure pump in fluid communication with a first and second riser tube, and at least one cross tube, and a control assembly including a pressure pump activation trigger configured to activate the pressure pump. The system further including a trailer hitch and a pivotable wheeled dolly configured to grip a portion of the one or more vehicle wash ramp and pivot about a wheeled axis, so as to raise at least a portion of the vehicle disinfection station off the ground for ease of transportation.

### RELATED APPLICATIONS

This application claims the benefit of U.S. Provisional Application 62/150,696, filed Apr. 21, 2015, and U.S. Provisional Application No. 62/157,657, filed May 6, 2015, each of which is hereby incorporated herein in its entirety by reference.

### TECHNICAL FIELD

The present disclosure relates generally to drive-over vehicle tire and undercarriage sanitizing systems. More particularly, the present disclosure relates to methods and systems for promoting safety and reducing contamination in vehicles after having transported or passed through areas potentially contaminated with a biological security threat.

### BACKGROUND

Avian influenza virus outbreaks over the recent years have put the application of current bio-security practices into question. One of the identified vectors for spreading the disease is the so called “mobile vector,” caused by vehicles and people moving into and out of areas where poultry is raised and processed. It is believed that more rigorous practices of vehicle hygiene will aid in the curtailing of future outbreaks.

Avian influenza viruses are influenza viruses that primarily infect birds, including poultry such as turkeys and chickens. Avian influenza viruses are type A influenza viruses that are genetically distinguishable from influenza viruses that usually infect people. There are many subtypes of avian influenza A viruses, including H7 and H5. Avian influenza viruses can be distinguished as “low pathogenic” and “high pathogenic” forms based on genetic features of the virus and the severity of the illness they cause in poultry.

Birds that are infected with avian influenza viruses are known to spread the virus through saliva, nasal secretions, and feces. Contact with feces or respiratory secretions may lead to the transmission of infection among poultry. Between flocks, infection usually spreads due to movement of infected birds and the actions of humans in moving feedstuff, personnel, equipment, and vehicles into and from premises that are contaminated with infected feces or respiratory secretions.

Infection among poultry often proves fatal and has the potential to decimate an entire flock within days. Even if not fatal, typically all other birds in proximity to the infected birds are destroyed for preventative measures. Further, given that the viruses can survive in many environments for weeks, federal and state regulations often prohibit the infected facilities from being reused for at least one month or more. This loss and delay can represent a serious financial toll on poultry growers and processors.

Over the years, various organizations have recommended that poultry growers improve their vehicle hygiene practices. Typically these practices involve maintaining separating traffic routes, so that only essential vehicles are permitted access to areas where birds are kept, as well as disinfecting the wheels and undercarriage of any vehicles entering these areas.

However, effectively disinfecting vehicles according to the recommended practices has been difficult to achieve. Commonly, hand-held spraying systems are employed to apply disinfectant to vehicles. Applying disinfectant through such a method can lead to non-uniform application; particularly to portions of the under carriage of the vehicle where application of disinfectant can be altogether missed. Non-uniformity in application can be even more prevalent when it is left up to drivers, with varying levels of biosecurity training, to disinfect their own vehicles. Moreover, proper disinfection of vehicles through these methods can be very time consuming.

Other vehicle wash systems, such as automatic car washes and wheel baths can be prohibitively expensive to small- and medium-sized poultry farms. Moreover, such systems are generally not mobile, in that they cannot easily be moved to another location once installed.

Accordingly, what is needed in the industry is an affordable, easy to use, mobile system for disinfecting vehicles to promote improved bio-security.

### SUMMARY OF THE DISCLOSURE

Embodiments of the present disclosure meet the need of the industry for an affordable, easy to use, mobile system for disinfecting vehicles to promote improved bio-security. In one embodiment, the system enables the automatic application of disinfectant in a quantity recommended by the manufacture substantially uniformly across the wheels and undercarriage of the vehicle. In one embodiment, the system requires minimal user interaction to reduce the likelihood of introducing human error during the application of disinfectant. In one embodiment, the system is portable or semi-portable, so that the system can be redeployed or readily moved from one location to another location in a short period of time. In one embodiment, the cost of the system is priced so that small- and medium-sized poultry farms can afford to purchase and install the system, thereby promoting more widespread bio-security practices across the agricultural industry.

One embodiment of the present disclosure provides a mobile vehicle disinfection system for promoting biosecurity through the application of a disinfectant to a vehicle transiting to and from an agricultural site. The mobile vehicle disinfection system can include a vehicle disinfection station, a wheeled moving dolly and a trailer hitch. The vehicle disinfection station can include a vehicle wash ramp, a disinfectant system, and a control assembly. The vehicle wash ramp can have a first ramp enabling the vehicle to smoothly transition from the ground to a vehicle wash platform and a second ramp enabling the vehicle to smoothly transition from the vehicle wash platform to the ground.

The vehicle wash ramp can include at least one channel formed therein. The disinfectant system can include a pressure pump in fluid communication with the first and second riser tube, and at least one cross tube. The first and second riser tubes can be in fluid connection with one another by the at least one cross tube, wherein the at least one cross tube at least partially resides within the at least one channel formed in the vehicle wash ramp, and wherein the first and second riser tubes and the at least one cross tube include a plurality of disinfectant dispensing nozzles. The control assembly can include a pressure pump activation trigger configured to activate the pressure pump, and a pressure pump deactivation trigger configured to deactivate the pressure pump.

The wheeled moving dolly can have a frame configured to grip a portion of the vehicle wash ramp and pivot about a wheel axis, so as to raise at least a portion of the vehicle disinfection station off the ground. The trailer hitch can be operably coupled to the vehicle wash ramp, and can be selectively coupleable to a vehicle for towing the mobile vehicle disinfection system from a first location to a second location.

One embodiment of the present disclosure provides an automatic vehicle disinfection station for promoting biosecurity through application of a disinfectant to a vehicle transiting to and from an agricultural site. In one embodiment, the automatic vehicle disinfection station includes two or more opposed vehicle wash ramps, a disinfectant supply system and a control assembly. In one embodiment, the two or more opposed vehicle wash ramps each include a first ramp enabling the vehicle to smoothly transition from the ground to a vehicle wash platform, and a second ramp enabling the vehicle to smoothly transition from the vehicle wash platform to the ground. Each vehicle wash platform of the two or more opposed vehicle wash ramps can include at least one channel formed therein.

In one embodiment, the disinfectant supply system can include a pressure pump in fluid communication with a first and second riser tube, and at least one cross tube. In some embodiments, the first and second riser tubes are fluidly coupled to one another by the at least one cross tube. In one embodiment, the at read this least one cross tube at least partially resides in the at least one channel formed in the vehicle wash platform. The first and second riser tubes and at least one cross tube can each include a plurality of disinfectant dispensing nozzles.

In one embodiment, the control assembly can include a pressure pump activation trigger configured to activate the pressure pump, and a pressure pump deactivation trigger configured to deactivate the pressure pump.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIGS. 1-2 are perspective views depicting an automatic vehicle disinfection station in accordance with an embodiment of the disclosure.

FIG. 3 is a top perspective view of a vehicle wash ramp in accordance with an embodiment of the disclosure.

FIG. 4 is a bottom perspective view of the vehicle wash ramp of FIG. 3.

FIG. 5 is a perspective view of two quick disconnect couplings, coupling a series of cross tubes in accordance with an embodiment of the disclosure.

FIG. 6 is a perspective view of two quick disconnect couplings, coupling a series of cross tubes to a riser tube in accordance with an embodiment of the disclosure.

FIGS. 7-8 are perspective views depicting an automatic vehicle disinfection station having a single vehicle wash ramp in accordance with an embodiment of the disclosure.

FIG. 9 is a perspective view of the bottom of an automatic vehicle disinfection station.

FIG. 10 is a perspective view of an automatic vehicle disinfection station with a trailer hitch in accordance with an embodiment of the disclosure.

FIG. 11 is a perspective view of an automatic vehicle disinfection system comprising a vehicle disinfection station, a trailer hitch and a wheeled moving dolly, wherein the wheeled dolly is supporting at least a portion of the vehicle disinfection station's weight on a plurality of wheels in accordance with an embodiment of the disclosure.

FIG. 12 is a perspective view of the automatic vehicle disinfection system of FIG. 11, wherein the wheeled moving dolly has been pivoted to lower the vehicle disinfection station to the ground.

While embodiments of the disclosure are amenable to various modifications and alternative forms, specifics thereof are shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure as defined by the appended claims.

### DETAILED DESCRIPTION

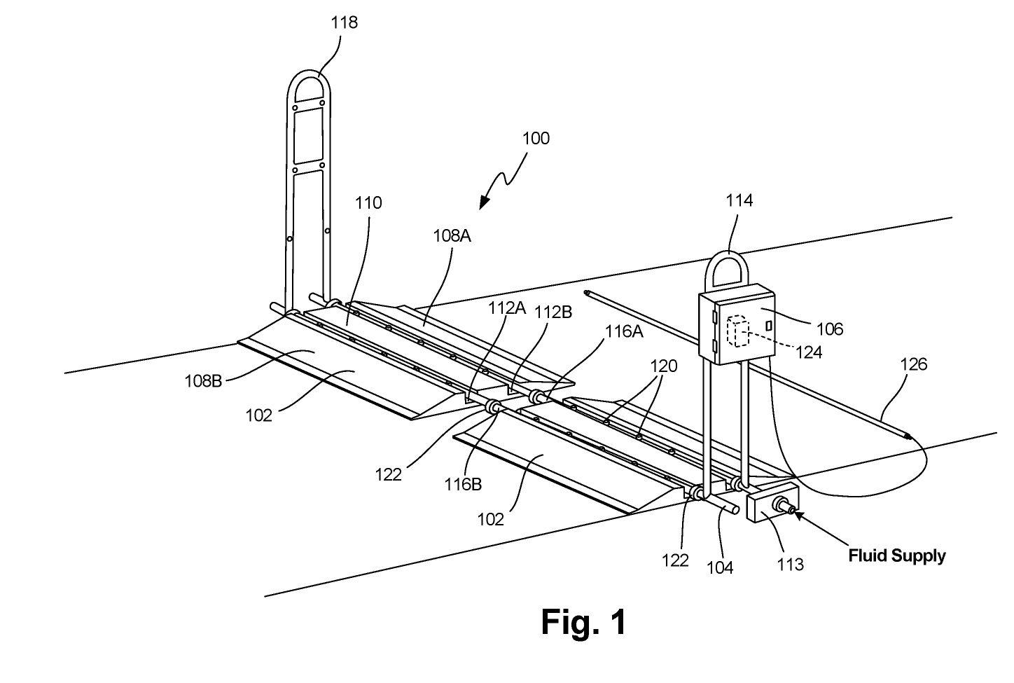

Referring to FIGS. 1-2, an automatic vehicle disinfection station 100 is depicted in accordance with an embodiment of the disclosure. Automatic vehicle disinfection station 100 generally includes one or more opposed vehicle wash ramps 102, a disinfectant supply system 104 and a control assembly 106.

Each vehicle wash ramp 102 can include a first ramp 108A enabling the vehicle to smoothly transition from the ground to a vehicle wash platform 110, and a second ramp 108B enabling the vehicle to smoothly transition from the vehicle wash platform 110 to the ground. In one embodiment, vehicle wash ramps 102 can be between one and four inches in height, and serve as a “speed bump,” thereby causing vehicles to slow as they pass over vehicle wash ramps 102. The vehicle wash platform 110 can include one or more channels 112A, 112B formed therein, wherein the channels 112A, 112B can be configured to receive a portion of the disinfectant supply system 104.

In one embodiment, the two or more vehicle wash ramps 102 are operably coupled to one another, thereby enabling the automatic vehicle disinfection station 100 to be set up to provide uniform coverage of the disinfectant to vehicles of varying widths, while at the same time enabling the automatic vehicle disinfection station 100 to be easily transported through manageably sized components.

In one embodiment, more than two vehicle wash ramps 102 can be coupled together to accommodate wider vehicles, or more than a single lane of traffic or vehicle at a given time. In one embodiment, more than two vehicle wash ramps 102 can be coupled together to improve portability by reducing the size of the individual vehicle wash ramps 102 to maximize the manageability of the individual components of the automatic vehicle disinfection station 100. In one embodiment, vehicle wash ramps 102 are between one and ten feet in width, as measured along the one or more channel 112A, 112B formed therein.

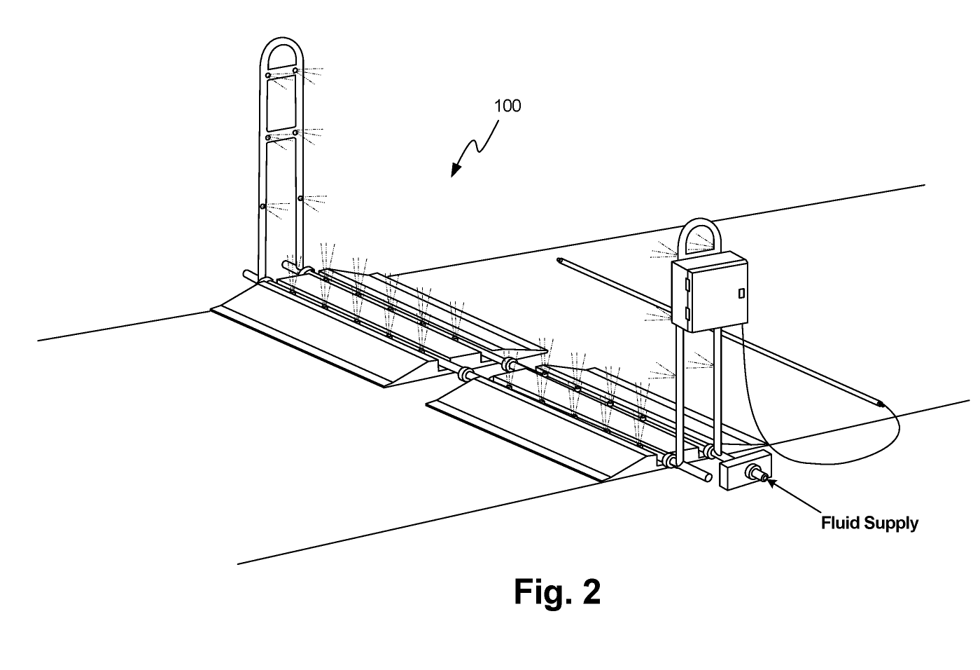

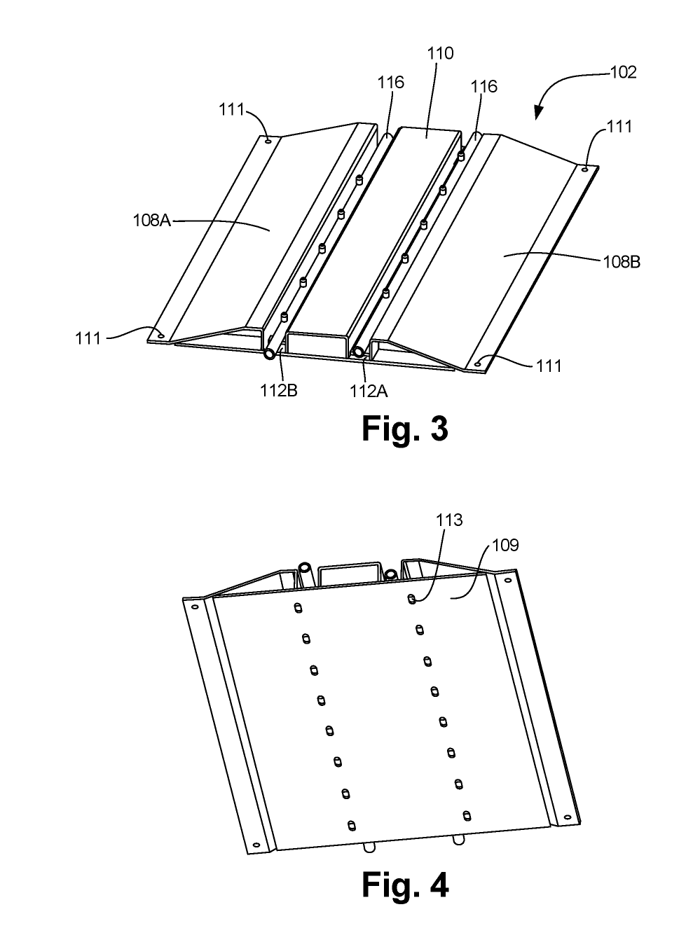

Referring to FIGS. 3-4, the top and bottom of a vehicle wash ramp 102 is depicted in accordance with an embodiment of the disclosure. The vehicle wash ramp 102 can be constructed of diamond plate, checker plate, tread plate, DURBAR floor plate, or some other type of texture surface plate on the top surface of the first ramp 108A, the vehicle wash platform 110 and the second ramp 108B. In one embodiment, the vehicle wash ramp 102 is constructed of a lightweight metal, such as stock steel, stainless steel or aluminum, with a regular pattern or lines on one side, with the reverse side being featureless. In other embodiments, the vehicle wash ramp 102 can be constructed of a lattice framework (not depicted) with or without support rails, thereby enabling debris being washed from the vehicle to fall to the ground below.

In one embodiment, the vehicle wash ramp 102 can include a base plate 109, configured to serve as a support for the other components of the vehicle wash ramp 102. Base plate 109 can be constructed of a metal plate. Base plate 109 can include a plurality of drains 113 positioned at the bottom of the one or more channel 112A, 112B, thereby permitting fluid residing within the one or more 112A, 112B, to properly drain. Such drains 113 can reduce corrosion of the vehicle wash ramp 102, as well as eliminating standing water.

In one embodiment, the first ramp 108A and the second ramp 108B can be constructed of a metal plate bent in a series of angles to create a ramp. In one embodiment, the vehicle wash platform 110 can be constructed of a metal plate bent in a series of angles to create a platform. The first ramp 108A, the second ramp 108B and the vehicle wash platform 110 can be operably coupled to the vehicle base plate 109, such as by welding and/or connection by a series of fasteners.

The vehicle wash ramps 102 can be anchored to the ground via an anchoring system, such as ground engaging ties, or the vehicle wash ramps 102 can be held in position relative to the ground by its own weight. In one embodiment, the first ramp 108A and the second ramp 108B can include a plurality of apertures 111 configured to accept one or more ground anchors to secure the vehicle wash ramp 102 to the ground.

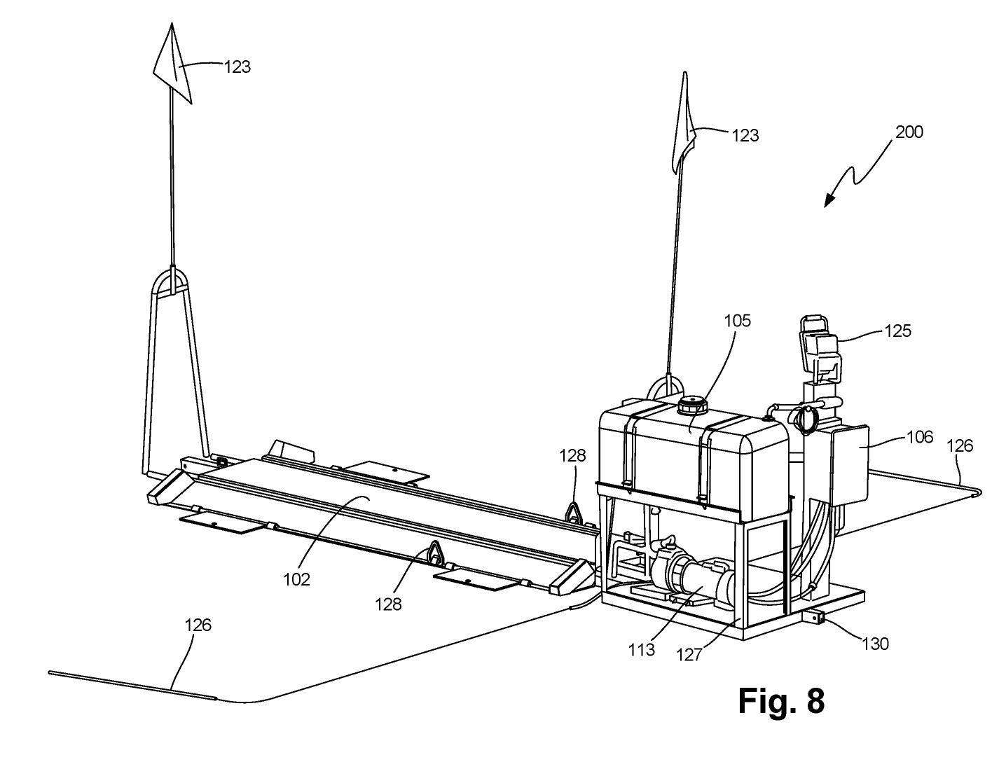

In one embodiment, the first ramp 108A and the second ramp 108B can include extension tabs 107A and 107B (as depicted in FIG. 7). Extension tabs 107A and 107B can include a plurality of apertures 111 configured to accept one or more ground anchors to secure the vehicle wash ramp 102 to the ground. In one embodiment, the extension tabs 107A and 107B are handedly coupled to the first ramp 108A and the second ramp 108B, so as to provide a smoother transition of the vehicle from the ground to the wash platform.

In one embodiment, the disinfectant supply system 104 can include a pressure pump 113, in fluid communication with a first riser tube 114, one or more cross tubes 116, and a second riser tube 118. In one embodiment, the first riser tube 114 and second riser tube 118 are in fluid communication with one another by at least one cross tube 116. In one embodiment, riser tubes 114 and 118 are U-shaped. In this embodiment, riser tubes 114 and 118 can fluidly connect to two cross tube 116 (as depicted in FIGS. 1-2). In other embodiments, there can be more than two cross tubes and the risers can be in other configurations, for example a multiple U-shaped configuration. In other embodiments, there can be more than two risers to accommodate more than a single lane of traffic or vehicle at a given time. In one embodiment, the one or more cross tubes 116 can be constructed of a flexible, or semi-flexible material to allow for deformations as vehicles pass over vehicle wash ramps 102. Such flexibility reduces breakage and wear of the cross tubes 160.

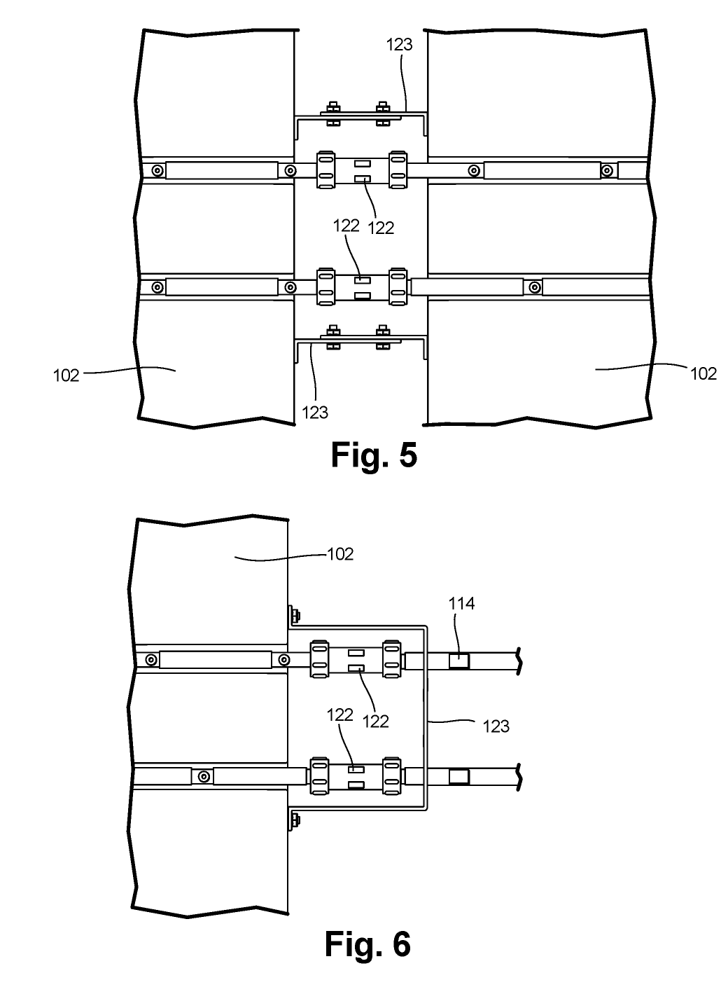

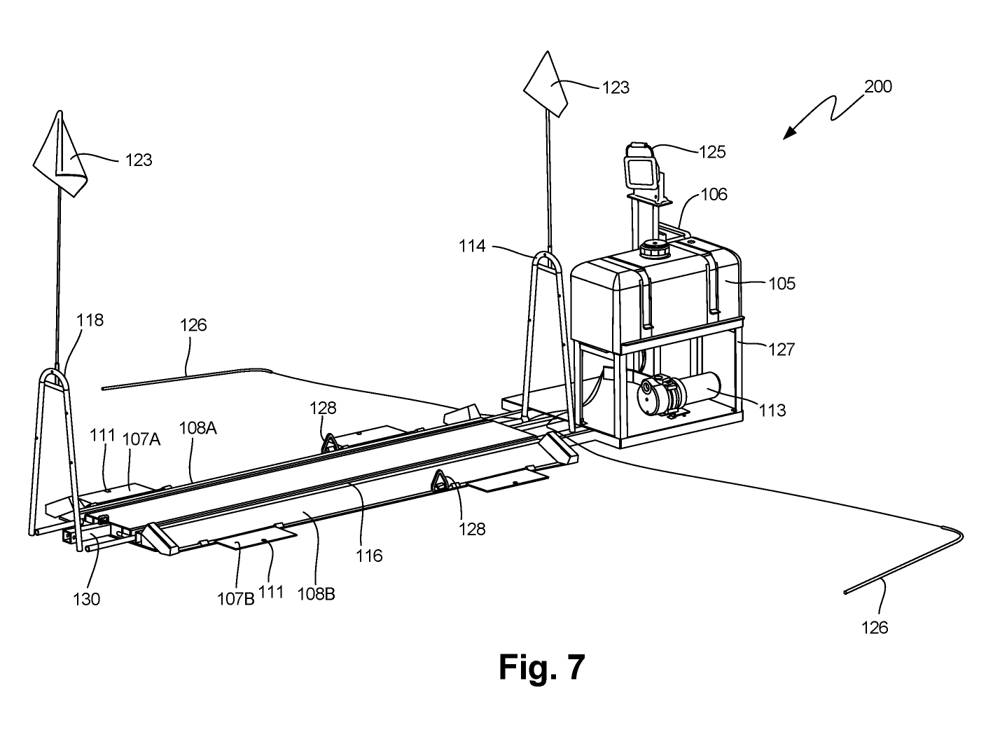

Referring to FIGS. 5-6, quick disconnect couplings 122 are depicted in accordance with an embodiment of the disclosure. In one embodiment, the various sections of disinfectant supply system 104, for example the various cross tubes 116, and riser tube 114, 118 are coupled to one another by quick disconnect couplings 122, thereby enabling vehicle disinfection station 100 to be more easily assembled and disassembled. In one embodiment, quick disconnect couplings 122 can be treaded couplings. In another embodiment, quick disconnect couplings 122 can be snap fit couplings. Quick disconnect couplings 122 can greatly improve functionality of the vehicle disinfection station 100 by reducing the amount of time in which the vehicle disinfection station 100 can be assembled and disassembled to promote mobility, as well as enable various portions of the disinfectant supply system 104 to be replaced with greater ease.

In one embodiment, one or more brackets 123 (depicted in FIGS. 5-6) can additionally be used to couple various portions of the vehicle disinfection station 100, including one or more vehicle wash ramps 102 and/or riser tubes 114/116 together. The one or more brackets 123 can be constructed of metal, and can be releasably coupled to the one or more vehicle wash ramps 102 and/or riser tubes 114/116 by a plurality of fasteners. In other embodiments, the one or more brackets 123 can be permanently mounted to a vehicle wash ramp 102 or riser tube 114/116 for selective engagement with other portions of the vehicle disinfection station 100.

The riser tubes 114 and 118, and cross tube 116 can include a plurality of disinfectant dispensing nozzles 120. In one embodiment, the dispensing nozzles 120 are evenly spaced across riser tubes 114 and 118, and cross tube 116 over the span of the width of the vehicle, thereby enabling both left and right tires and undercarriage surfaces to be properly sanitized. In other embodiments, dispensing nozzles 120 are spaced in a manner that provides more coverage to areas of the vehicle most likely to be in contact with bio-hazardous material. In one embodiment, a plurality of dispensing nozzles 120 are configured in a variety of locations at different angles thereby enabling all surfaces of the vehicle to receive ample coverage of disinfectant. In one embodiment, dispensing nozzles 120 are configured to dispense disinfectant to the wheel inners, outers, treads, wheel arches, undercarriage, front, rear, sides, and roof top (optional). In one embodiment, dispensing nozzles 120 disperse disinfectant at a wide pattern angle, such as a pattern angle between ten and sixty degrees. In one embodiment, the dispensing nozzles 120 pattern angle is approximately forty degrees. In one embodiment, at least a portion of cross tube 116A, 116B at least partially resides in channel 112A, 112B, thereby enabling the dispensing nozzles 120 to be protected from vehicles moving over vehicle wash ramps 102.

In one embodiment, the disinfectant supply system 104 further includes a tank 105 (depicted in FIGS. 7-9) configured to store a disinfecting agent or components thereof. The pressure pump 113 can be a high pressure pump configured to boost the pressure of the disinfectant sufficiently to enable delivery of the required amount of disinfectant in the amount prescribed by the manufacturer of the disinfectant over a time period of less than one minute.

In one embodiment, the control assembly 106 can include a pressure pump activation trigger 126 configured to activate pressure pump 113, and a pressure pump deactivation trigger 124 configured to deactivate pressure pump 113. In one embodiment, pressure pump activation trigger 126 can incorporate an air switch, configured to activate pressure pump 113 upon being driven over by a vehicle. In other embodiments, pressure pump activation trigger 126 can be a push button start activated by a user, a photo electric eye configured to activate pressure pump 113 upon the sensing of a vehicle, or a combination of the above mentioned triggers.

In one embodiment, pressure pump deactivation trigger 124 can be a timer configured to deactivate pressure pump 113 upon the passage of predetermined amount of time after the triggering of pressure pump activation trigger 126. In other embodiments, pressure pump activation trigger 126 can be photo electric eye configured to deactivate pressure pump 113 upon ceasing to sense a vehicle, or a combination of the above mentioned triggers. In one embodiment, the control assembly 106 includes a countdown timer visible to the driver, thereby enabling the driver to adjust the speed of their vehicle to ensure substantially equal disinfectant application over the entire length of the vehicle.

In operation, a location for positioning of the vehicle disinfection station 100 can be determined. The one or more vehicle wash ramps 102 and riser tubes 114, 118 can be transported to the location, and laid out in the appropriate configuration for assembly. In some embodiments, the one or more vehicle wash ramps 102 and/or riser tubes 114, 118 can be coupled together via one or more brackets 123.

The components of the disinfectant supply system 104 and the control assembly 106 can be installed and fitted within the one or more vehicle wash ramps 102 and/or riser tubes 114, 118 as required. In particular, the at least one cross tube 116 can be positioned within channel 112 of the one or more vehicle wash ramps 102 and can be coupled to the riser tubes 114, 118. In some embodiments, several cross tubes 116 are required to cross the span of the roadway. In such embodiments, the cross tubes 116 can be coupled together via one or more quick disconnects 122. One or more quick disconnects 122 can also be used to couple the cross tubes 116 with the riser tubes 114, 118.

The one or more pressure pump activation triggers 122 can be installed and/or positioned appropriately in front of or behind the vehicle disinfection station. Tank 105 can be filled with disinfectant, and the vehicle disinfection station 100 can be coupled to an electrical and/or water supply.

It should be understood that the individual steps used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

Referring to FIGS. 7-8, an automatic vehicle disinfection station 200 having a single vehicle wash ramp 102 is depicted in accordance with an embodiment of the disclosure. The automatic vehicle disinfection station 200 can include all of the components of automatic vehicle station 100, with the exception that automatic vehicle disinfection station 200 generally includes only a single vehicle wash ramp 102.

In one embodiment, automatic vehicle disinfection station 200 includes one or more flags 123, configured to improve positional awareness of the riser tubes 114 and 118, and other components of the automatic vehicle disinfection station 200 to drivers of vehicles using the automatic vehicle disinfection station 200.

In one embodiment, the automatic vehicle disinfection station 200 can further include a light 125 to selectively illuminate the vehicle disinfection station 200. In some embodiments, the light 125 is turned on and off by the one or more pressure pump activation and or deactivation triggers 122, such as an air switch, push button start, photo electric eye, timer, combination thereof. In one embodiment, the light 125 can be positioned on top of the control assembly 106. In one embodiment, light 125 can include a countdown timer displaying the number of seconds remaining for the application of disinfectant, and configured to be visible to the driver, so as to enable the driver to adjust the speed of their vehicle to ensure substantially equal disinfectant application over the entire length of the vehicle.

In one embodiment, the tank 105 can be supported by a frame structure 127. Frame structure 127 can be configured to raise tank 105 off the ground, so as to enable gravity to assist drainage of tank 105 during operation. Frame structure 127 can also be configured to house the pressure pump 113, as well as other components of the disinfectant supply system 104 and control assembly 106 both during and after transport.

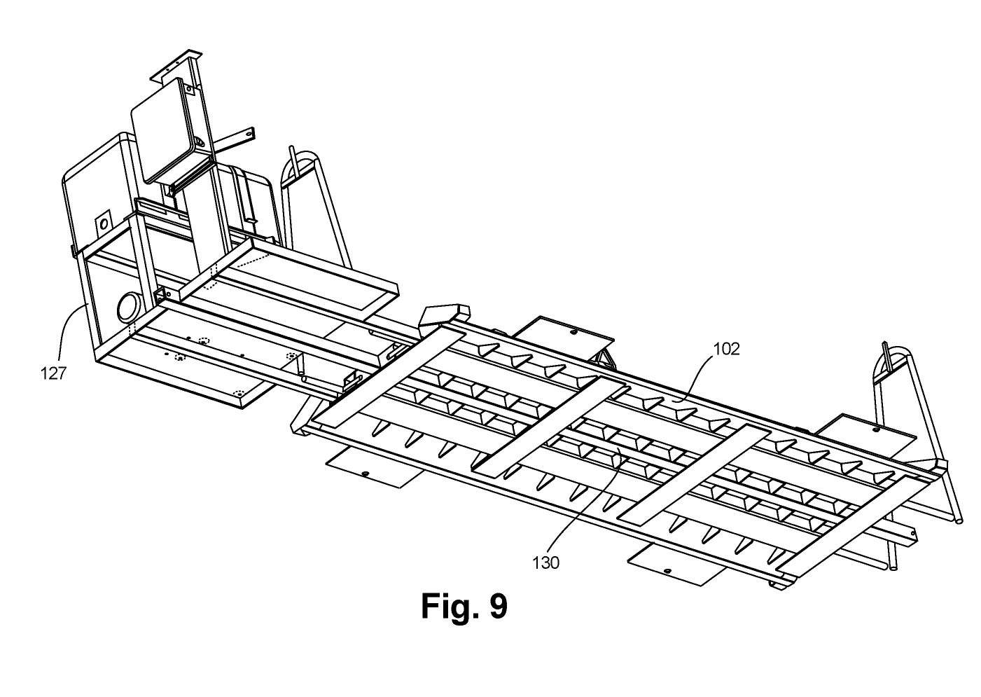

Additionally, automatic vehicle disinfection station 200 can include a rigid frame member 130. As depicted in FIG. 9, the rigid frame member 130 can be positioned on the bottom of the vehicle disinfection station, and can extend the full length of the vehicle disinfection station 200. Rigid frame member 130 can be configured to provide a rigid support for the various components of the automatic vehicle disinfection station 200, particularly during transport. For example, the vehicle wash ramp 102, frame structure 127, and other portions of the of the vehicle disinfection station 200 can be operably coupled to the rigid frame member 130, so as to utilize the rigid frame member 130 as the foundation of the vehicle disinfection station. As depicted in FIG. 9, the vehicle disinfection station 200 can additionally include a plurality of other supports operably coupled to the rigid frame member 130.

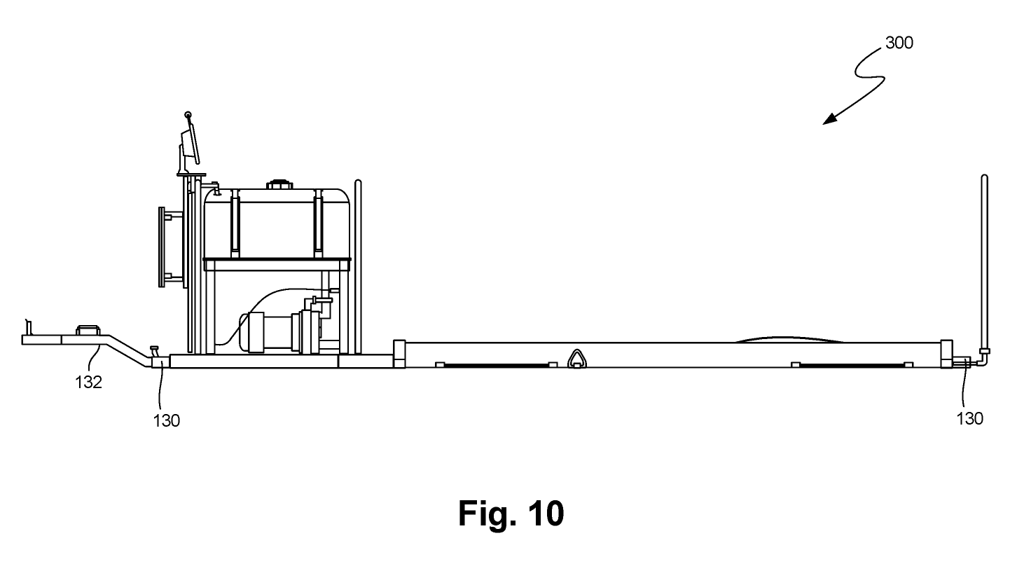

In one embodiment, the rigid frame member 130 can be a tubular metal structure. As depicted in FIG. 10, in one embodiment the rigid frame member 130 can be configured to receive a trailer hitch 132. Trailer hitch 132 can be operably coupled to, for example a tow ball, of a vehicle for ease in transporting the vehicle disinfection station 200 from one location to another.

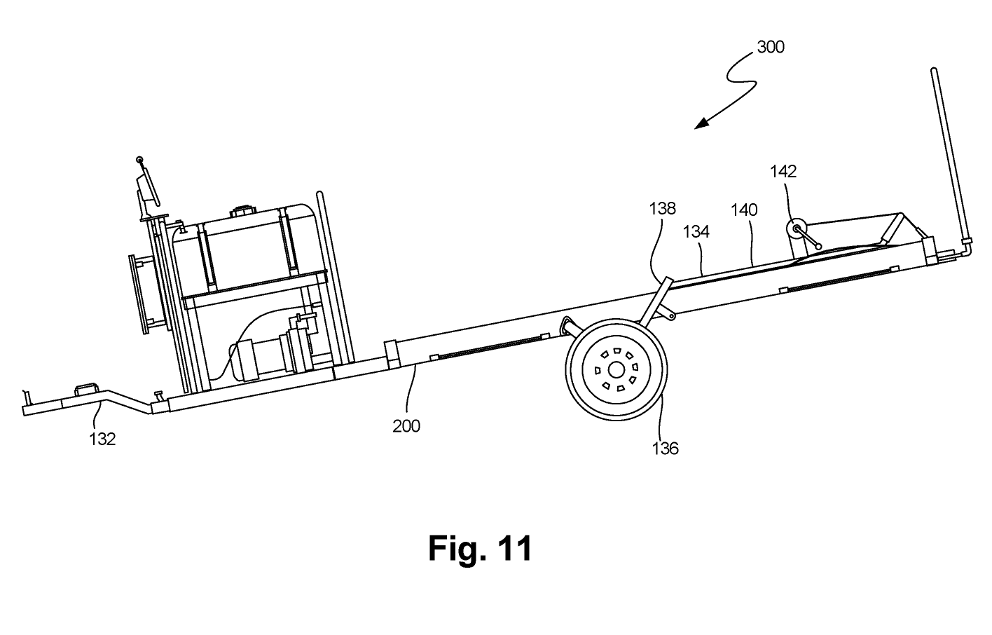

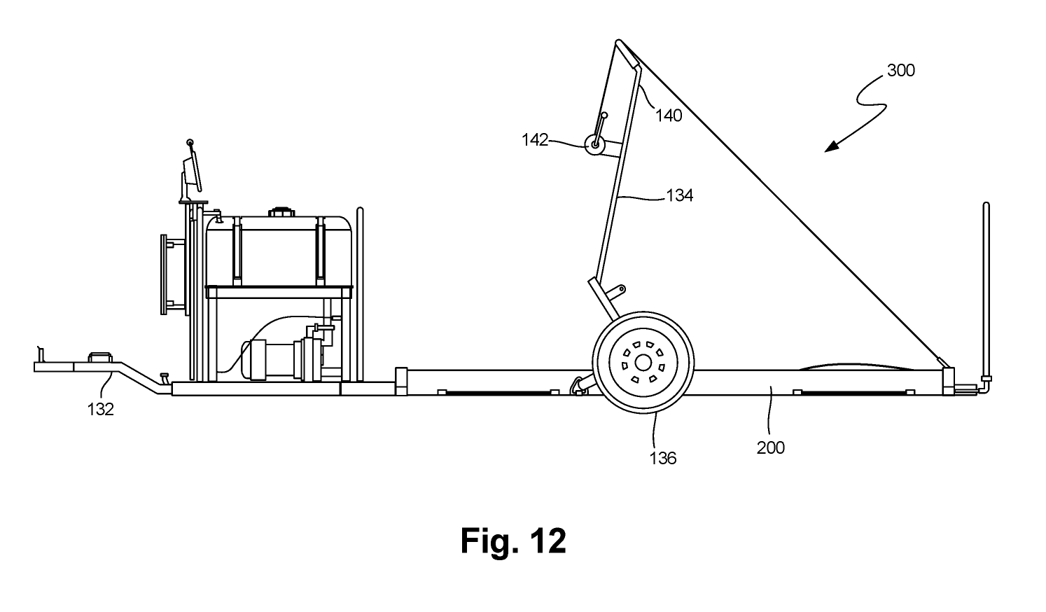

Referring to FIGS. 11-12, an automatic vehicle disinfection system 300 including the automatic vehicle disinfection station 200 is depicted in accordance with an embodiment of the disclosure. In one embodiment, the automatic vehicle disinfection system 300 includes the automatic vehicle disinfection system 200, a wheeled moving dolly 134 and a trailer hitch 132. In one embodiment, wheeled moving dolly 134 can include a plurality of wheels 136, a frame structure 138 configured to support at least a portion of the automatic vehicle disinfection station 200, and an arm 140 including a winch 142, configured to secure the moving dolly 134 to a portion of the automatic vehicle disinfection station 200.

In operation, the automatic vehicle disinfection station 200 can be towed into position by a vehicle. The automatic vehicle disinfection station 200 can be coupled to the vehicle via the trailer hitch 132. Once positioned, the trailer hitch 132 can be disconnected from the vehicle. If desired, the trailer hitch 132 can be removed from the rigid frame member 130.

The automatic vehicle disinfection station 200 can be separated from the moving dolly 134 by unwinding cable from the winch 142, thereby causing the moving dolly 134 to pivot under gravity such that the vehicle disinfection station 200 is lowered to the ground. Once the vehicle disinfection station 200 has been fully lowered to the ground, the wench 142 cable can be disconnected from the vehicle disinfection station 200, and the moving dolly 134, which can be configured to grip a portion of the vehicle wash ramp 102, can be released from the vehicle disinfection station 200.

One or more pressure pump activation and/or deactivation triggers 126 can be installed and/or positioned appropriately in front of or behind the vehicle disinfection station. Tank 105 can be filled with disinfectant, and the vehicle disinfection station 200 can then be coupled to an electrical and/or water supply.

In one embodiment, the automatic vehicle disinfection station 200 is fully assembled, such that no assembly is required, besides connection to an electrical/water source, upon positioning the vehicle disinfection station.

It should be understood that the individual steps used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

Persons of ordinary skill in the relevant arts will recognize that embodiments may comprise fewer features than illustrated in any individual embodiment described above. The embodiments described herein are not meant to be an exhaustive presentation of the ways in which the various features may be combined. Accordingly, the embodiments are not mutually exclusive combinations of features; rather, embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Moreover, reference in the specification to “one embodiment,” “an embodiment,” or “some embodiments” means that a particular feature, structure, or characteristic, described in connection with the embodiment, is included in at least one embodiment of the teaching. The appearances of the phrase “in one embodiment” in various places in the specification are not necessarily all referring to the same embodiment.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

What is claimed is:

1. A mobile vehicle disinfection system for promoting biosecurity through the application of a disinfectant to a vehicle transiting to and from an agricultural site, the system comprising:
a vehicle disinfection station, including—
a vehicle wash ramp having a first ramp enabling the vehicle to smoothly transition from the ground to a vehicle wash platform and a second ramp enabling the vehicle to smoothly transition from the vehicle wash platform to the ground, wherein the vehicle wash ramp includes at least one channel formed therein;
a disinfectant system including a pressure pump in fluid communication with the first and second riser tube, and at least one cross tube, wherein the first and second riser tubes are in fluid connection with one another by the at least one cross tube, wherein the at least one cross tube at least partially resides within the at least one channel formed in the vehicle wash ramp, and wherein the first and second riser tubes and the at least one cross tube include a plurality of disinfectant dispensing nozzles; and
a control assembly including a pressure pump activation trigger configured to activate the pressure pump, and a pressure pump deactivation trigger configured to deactivate the pressure pump;
a wheeled moving dolly having a frame configured to grip a portion of the vehicle wash ramp and pivot about a wheel axis, so as to raise at least a portion of the vehicle disinfection station off the ground;
a trailer hitch operably coupled to the vehicle wash ramp, the trailer hitch selectively coupleable to a vehicle for towing the mobile vehicle disinfection system.

2. The mobile vehicle disinfection system of claim 1, wherein the pressure pump activation trigger is at least one of an air switch, pushbutton, photoelectric eye, or combination thereof.

3. The mobile vehicle disinfection system of claim 1, wherein the pressure pump deactivation trigger is at least one of an air switch, photoelectric eye, timer, or combination thereof.

4. The mobile vehicle disinfection system of claim 1, wherein the vehicle wash ramp measures approximately eight feet in width.

5. The mobile vehicle disinfection system of claim 1, further comprising one or more additional vehicle wash ramps to increase the width of the vehicle disinfection station.

6. The mobile vehicle disinfection system of claim 1, further comprising a countdown timer displaying the number of seconds remaining for the application of disinfectant, and configured to be visible to the driver, so as to enable the driver to adjust the speed of their vehicle to ensure substantially equal disinfectant application over the entire length of the vehicle.

7. The mobile vehicle disinfection system of claim 1, wherein the at least one cross tube couples to other components of the disinfectant system via one or more quick disconnect couplings.

8. The mobile vehicle disinfection system of claim 1, wherein the plurality of disinfectant dispensing nozzles are evenly spaced across the riser tubes and cross tubes substantially over the span of a standard vehicle width, thereby enabling both left and right tires and undercarriage surfaces of the vehicle to be properly sanitized.

9. The mobile vehicle disinfection system of claim 1, wherein the plurality of disinfectant dispensing nozzles are spaced in a manner that provides more coverage to areas of the vehicle most likely to be in contact with bio-hazardous material.

10. The mobile vehicle disinfection system of claim 1, wherein the plurality of disinfectant dispensing nozzles are configured to dispense disinfectant to at least one of the wheel inners, outers, treads, wheel arches, undercarriage, front, rear, and sides of the vehicle.

11. An automatic vehicle disinfection station for promoting biosecurity through application of a disinfectant to a vehicle transiting to and from an agricultural site, the system comprising:
a pair of opposed vehicle wash ramps, where each vehicle wash ramp includes a first ramp enabling the vehicle to smoothly transition from the ground to a vehicle wash platform, and a second ramp enabling the vehicle to smoothly transition from the vehicle wash platform to the ground, wherein the vehicle wash platform includes at least one channel formed therein;
a disinfectant supply system including a pressure pump in fluid communication with a first and second riser tube, and at least one cross tube, wherein the first and second riser tubes are fluidly coupled to one another by the at least one cross tube, wherein at least one cross tube at least partially resides in the at least one channel formed in the vehicle wash platform, and wherein the first and second riser tubes and at least one cross tube include a plurality of disinfectant dispensing nozzles; and
a control assembly including a pressure pump activation trigger configured to activate the pressure pump, and a pressure pump deactivation trigger configured to deactivate the pressure pump.

12. The mobile vehicle disinfection system of claim 1, wherein the pressure pump activation trigger is at least one of an air switch, pushbutton, photoelectric eye, or combination thereof.

13. The mobile vehicle disinfection system of claim 1, wherein the pressure pump deactivation trigger is at least one of an air switch, photoelectric eye, timer, or combination thereof.

14. The mobile vehicle disinfection system of claim 1, wherein the vehicle wash ramp measures approximately eight feet in width.

15. The mobile vehicle disinfection system of claim 1, further comprising a countdown timer configured to be visible to the driver, so as to enable the driver to adjust the speed of their vehicle to ensure substantially equal disinfectant application over the entire length of the vehicle.

16. The mobile vehicle disinfection system of claim 1, wherein the at least one cross tube couples to other components of the disinfectant system via one or more quick disconnect couplings.

17. The mobile vehicle disinfection system of claim 1, wherein the plurality of disinfectant dispensing nozzles are evenly spaced across the riser tubes and cross tubes substantially over the span of a standard vehicle width, thereby enabling both left and right tires and undercarriage surfaces of the vehicle to be properly sanitized.

18. The mobile vehicle disinfection system of claim 1, wherein the plurality of disinfectant dispensing nozzles are spaced in a manner that provides more coverage to areas of the vehicle most likely to be in contact with bio-hazardous material.

19. The mobile vehicle disinfection system of claim 1, wherein the plurality of disinfectant dispensing nozzles are configured to dispense disinfectant to at least one of the wheel inners, outers, treads, wheel arches, undercarriage, front, rear, and sides of the vehicle.
